(19) United States
(12) Patent Application Publication
(10) Pub. No.: US 2013/0274619 A1
(43) Pub. Date: Oct. 17, 2013

(54) SYSTEMS AND METHODS FOR A LOW-PROFILE VASCULAR PRESSURE MEASUREMENT DEVICE

(71) Applicant: SensorCath, Inc., (unknown)

(72) Inventors: Robert T. Stone, Sunnyvale, CA (US); Jeffrey J. Christian, Morgan Hill, CA (US); Darius Adam Przygoda, Mountain View, CA (US); Tat-Jin Teo, Santa Clara, CA (US)

(21) Appl. No.: 13/840,505

(22) Filed: Mar. 15, 2013

Related U.S. Application Data
(63) Continuation-in-part of application No. 13/664,357, filed on Oct. 30, 2012.
(60) Provisional application No. 61/554,227, filed on Nov. 1, 2011.

(30) Foreign Application Priority Data
Oct. 31, 2012 (US) PCT/US2012/062777

(51) Int. Cl. A61B 5/00; A61B 5/0215

(52) U.S. Cl. CPC A61B 5/6851; A61B 5/0215; A61B 5/0004
USPC 600/488

(57) ABSTRACT

A measuring system includes an elongated sleeve having a compact diameter configured to be delivered over a standard guide wire and able to be flexibly threaded into a tortuous or diseased vascular pathway of a human. Sensor(s), located at a distal end of the sleeve, measure physiological parameter(s) inside the human. The sleeve measuring system has an outer diameter of approximately 20 mils or less and is capable of accommodating the standard guide wire, typically 14 mils in diameter.

### CROSS REFERENCE TO RELATED APPLICATIONS

This application is a continuation-in-part of and claims priority to U.S. application Ser. No. 13/664,357 filed on Oct. 30, 2012, entitled “Systems and Methods for a Wireless Vascular Pressure Measurement Device”, which application claims priority to U.S. Provisional Application No. 61/554,227 filed on Nov. 1, 2011, entitled “Systems and Methods for a Wireless Vascular Pressure Measurement Device”, which are hereby fully incorporated by reference.

### BACKGROUND

The present invention relates to a system and methods for a vascular pressure measurement device.

Pressure wire has been used in the catheterization laboratory as part of the Percutaneous Coronary Intervention (PCI) procedure since the late 1980's. The form factor most commonly used is that of the 0.014″ diameter guide wire.

A typical construction of a pressure wire involves a radio opaque spring tip in the distal end, a housing or holder for the pressure sensor itself a few centimeters proximal to the distal end and a lumen, which is a hollow channel, to accommodate the electrical conductors or optical fibers depending on whether the pressure sensor is electrical or optical in its theory of operation.

At the proximal end where the pressure wire exits the human body, an electrical interface is typically provided for signal acquisition, processing and display. Some user input interface can also be provided.

There are times the pressure wires are used like a guide wire on which other interventional device like balloon or stent deployment system can be delivered. Consequently, the profile of a pressure wire needs to be maintained throughout the length of the body of the pressure wire. This requirement also applies to the electrical contacts where the above electrical interface for acquisition is located. Having electrical contacts that remain flushed with the pressure wire body profile is therefore important.

The electrical interface where the pressure signal is acquired and/or processed also needs to be removable when the pressure wire is to be used as a guide wire for delivery of other interventional devices.

Some clinicians, for tactile familiarity, have a preference to use a particular guide wire to begin the interventional procedure. These guide wires are also referred to as the primary guide wires. If a separate pressure wire is used for subsequent pressure measurement, it would then involve a wire exchange step which is sometimes undesirable especially if it is a very difficult lesion, a narrowing or obstruction, in the vessel, to cross the first time.

It would then be preferable to measure the pressure with a catheter over the guide wire that is already in place. The disadvantage is that the accuracy of the pressure measurement relative to that from a pressure wire might be reduced due to the presence of the catheter. It is therefore important to have a micro-catheter as small as possible.

The trade off between measuring the pressure in the form of a guide wire or a stand-alone micro-catheter will be discussed when the present invention is further described below.

While the sensing technology continues to make progress in terms of sensor miniaturization and improved processing and manufacturing method can achieve better performance and cost, many limitations remain.

Some of the limitations of prior art pressure wire are described here.

It is common to have a lumen in the region proximal to the sensor to accommodate the electrical or optical transmission lines. Unfortunately, this reduces somewhat the ability to provide a 1:1 torque transmission from the proximal end to the distal end of the pressure wire. Consequently, many physicians tend to use their preferred guide wire to cross the lesion in the vessel and only when they want to perform pressure measurement, they would do a wire exchange to deploy a pressure wire.

A culprit lesion that is responsible for the symptoms that bring the patient into the catheterization laboratory in the first place is often times one that has a severe narrowing of the vessel lumen. Many physicians may see no need to further measure the pressure gradient caused by that culprit lesion to assess its hemo-dynamic significance. In addition, it would be challenging to deploy a pressure wire there since it usually will not perform as well as one designed to be a primary guide wire.

On the other hand, if there are multiple lesions, one may appear to be only marginally constrictive from the appearance of the angiogram. The decision to intervene will then be based upon the hemo-dynamic of the lesion and pressure gradient measurement will be very helpful.

The pressure wire is also tethered to a non-sterile electronic equipment which as described above will acquire and process the signal from the sensor. The electronic equipment typically will also have a user input device to facilitate the procedure and provide a display for the signal as well as any processed results.

This need for electronic equipment near the sterile field in the catheterization laboratory can impede a smooth work flow in the catheterization laboratory. One solution is to have the electrical interface located far enough from the sterile field to avoid accidental contamination. However, this arrangement comes at the expense of degraded signal quality due to the parasitic noise induced by the extended connection length.

U.S. Pat. No. 7,724,148 “Transceiver Unit in a Pressure Measurement System” by Samuelsson et al., which is incorporated by reference for all purposes, provides a wireless interface which is attached at the proximal end of the pressure wire. Pressure signals are processed and transmitted from the proximal end of the pressure wire wirelessly to a wireless receiver in the non-sterile area. The size is such that while it can function as a handle for the pressure wire, it is too large to function adequately like a torque device, known sometimes as a torquer, commonly used to manipulate a 0.014″ guide wire.

The position of the wireless transceiver is also fixed by the location of the electrical contacts on the pressure wire and would not allow the operator to manipulate the guide wire in a way that is similar to a torque device. A regular torque device can be used at an arbitrary position along the proximal region of the guide wire according to personal preference and the requirement of the anatomy involved at the procedure.

Implementing the wireless transceiver in the form factor of a torque device allows it to move to a location along the guide wire closer to where it enters the touhy borst. This will allow better control of the wire movement.

With prior art pressure wire, it is common to have only a single sensor at the distal tip as described above. In some procedure, it is desired to measure both the pressure distal to the lesion in a coronary vessel as well as the pressure in the aorta, the ratio of which is a useful ratio to estimate a parameter known as Fractional Flow Reserve.

This desire to measure pressures at two locations requires a pullback operation to move the sensor from a location distal to a lesion in a coronary vessel to a location proximal to the lesion. Having multiple sensors would typically increase the number of transmission lines and can be a difficult task given the small space of a guide wire form factor.

It is therefore apparent that an urgent need exists for an improved pressure measuring device that includes one or more of the following improvements: (i) elimination of the hollow lumen in the body of the guide wire, (ii) wireless transmission, (iii) multiple sensors and (iv) stand-alone low-profile micro-catheter compatible with primary guide wires, resulting in better handling characteristics, better measurements, and shortened invasive procedures.

### SUMMARY

To achieve the foregoing and in accordance with the present invention, a system and method for measuring at least one physiological parameter of at least one location inside a human is provided. In particular, a wireless vascular pressure measurement device for measuring parameter(s) at one or more vascular locations inside a human is provided.

In one embodiment, a vascular system includes an elongated sleeve and one or more sensors. The elongated sleeve has a compact diameter configured to be delivered over a standard guide wire and further configured to be flexibly threaded into a tortuous or a diseased vascular pathway of a human. The sensor(s) can be located at a distal end of the sleeve, and configured to measure physiological parameter(s) at location(s) inside the human. In this embodiment, the sleeve has an outer diameter that is approximately 2.5 mils or less.

In some embodiment, the sleeve is formed by winding a planar layered structure into a helical construct. The sleeve may be formed by depositing a sequence of layers on a mandrel. The sequence of layers may include alternating conductive and insulating layers.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 is a schematic showing the key components making up a pressure wire measurement system;

FIG. 2 is a schematic showing the conductors between the sensor and proximal electrical contacts in a prior art embodiment;

FIG. 3 illustrates one preferred embodiment of the electrically conductive structures of the present invention;

FIG. 4 illustrates the cross-sectional view of FIG. 3;

FIGS. 5a, b, c and d illustrate a torque device in accordance to one embodiment of the invention;

FIG. 6 illustrates one preferred embodiment of the pressure wire to provide a guiding mechanism so that the torque device will engage the conductive traces at the appropriate orientation;

FIG. 7 illustrates another preferred embodiment of the pressure wire measurement system where there are two sensors deployed on a sleeve that can be delivered over a traditional guide wire, 110, not shown, and a torque device can wirelessly activate the sensors and shows the results from the signals return by these two sensors;

FIG. 8 illustrates another embodiment where the stand alone sleeve catheter with two sensors is in a rapid exchange catheter configuration with guide wire, 110, and a catheter handle, 810, now serving as the display for either the waveforms from the two sensors or the results after processing of the two waveforms or both, depending on the display size available. Two switches to control the electronics in the handle are also shown in this illustration;

FIG. 9 illustrates a planar structure wherein a dielectric layer is coated with a conductive layer which in turn is coated by an insulating layer;

FIG. 10 illustrates a planar layered structure wherein a dielectric layer is coated with a conductive layer which in turn is coated by an insulating layer. On the other side of the dielectric layer a second conductive layer is added;

FIG. 11 illustrates a layered construct wherein a second insulating layer is added to the dielectric layer before adding the conductive layer in the form of a conducting trace;

FIG. 12 provides a top view for the layered construct;

FIG. 13 illustrates a helical construct build up form a layered strip that is wound in a helical fashion;

FIG. 14 illustrates a layered structure whereby an inserted insulating layer is deposited on the dielectric layer before depositing the conductive layer; and

FIG. 15 illustrates constructing a sleeve by depositing a sequence of layers on a mandrel.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

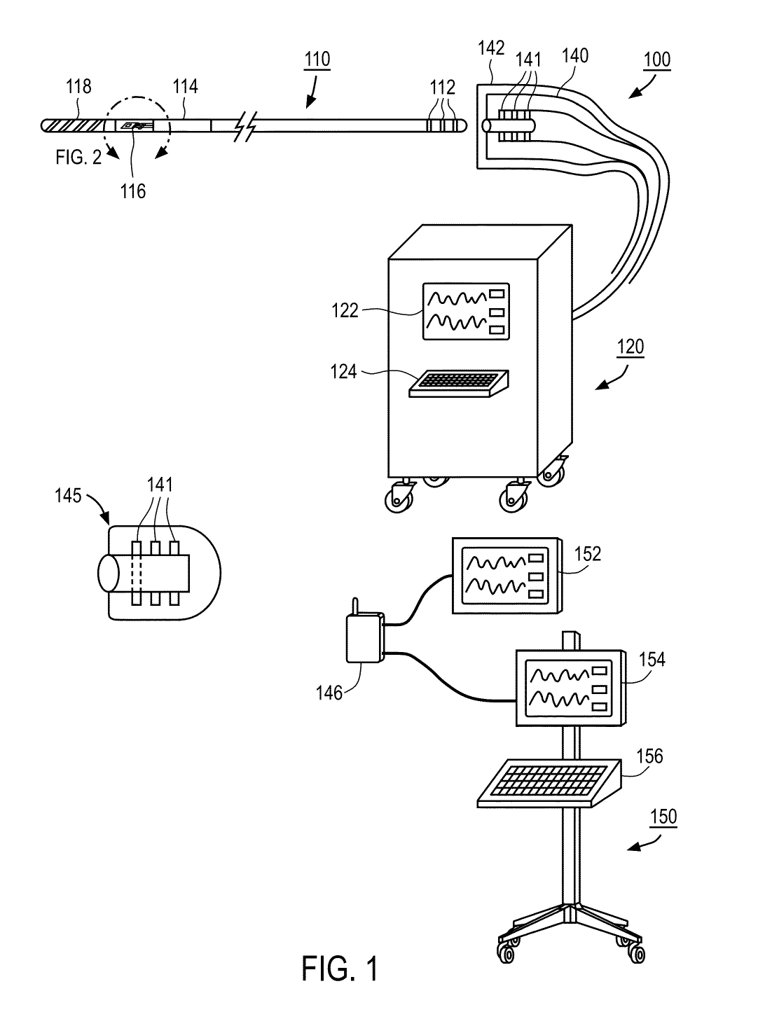

FIG. 1 shows one embodiment of a pressure wire measurement system, 100, not to scale. It includes a pressure wire, 110. The distal end, designated 118, is usually radio-opaque to allow for visualization under X-ray and is usually implemented as a coil to make it floppy and atraumatic. The pressure sensor is designated 116 and is often followed by another coil section 114 for desired stiffness. The remaining body of the pressure wire often has a hollow lumen to accommodate the electrical transmission lines (not shown) connecting the sensor 116 with the electrical contacts 112 at the proximal end.

The hollow lumen in the proximal portion of the pressure wire designed to accommodate the electrical or optical transmission conductors reduces the fidelity of the torque transmission due to the reduced rigidity of the body of the pressure wire. System 100 addresses this issue by having thin conductive traces on the central core wire.

FIG. 1 also shows a connector 140 that couples to the proximal end of the pressure wire 110. Internal to connector 140, there are electrical contacts 141 that mate with the counterpart 112 on the pressure wire. The connector 140 being non-sterile needs to be enclosed with a sterile barrier 142, typically a sterile bag, to prevent contamination of the sterile field during the PCI procedure.

It is also possible to have a long pressure wire such that the connect 140 is far remove from the sterile field where the risk of contamination is low and a sterile barrier 142 may not be needed. However, if long transmission lines are used as a consequence of having a long pressure wire, signal quality may be degraded.

The connector 140 is coupled to an electronic equipment 120, where the signals from the sensor can be acquired, processed and display with the display 122. If user input is needed, an input device 124 can also be located on the electronic equipment 120.

In another embodiment, a wireless implementation is described. In this embodiment, a wireless transceiver 145 is coupled to the pressure wire such that the electrical contacts 141, in the transceiver 145, mates with the electrical contact 112 on the pressure wire 110. The signals are then wirelessly received by a wireless transceiver 146 which can then display the information on a display 152 or couple to the electronic equipment 150 which may take the form of an Intravenous pole with a display 154 and an input device 156.

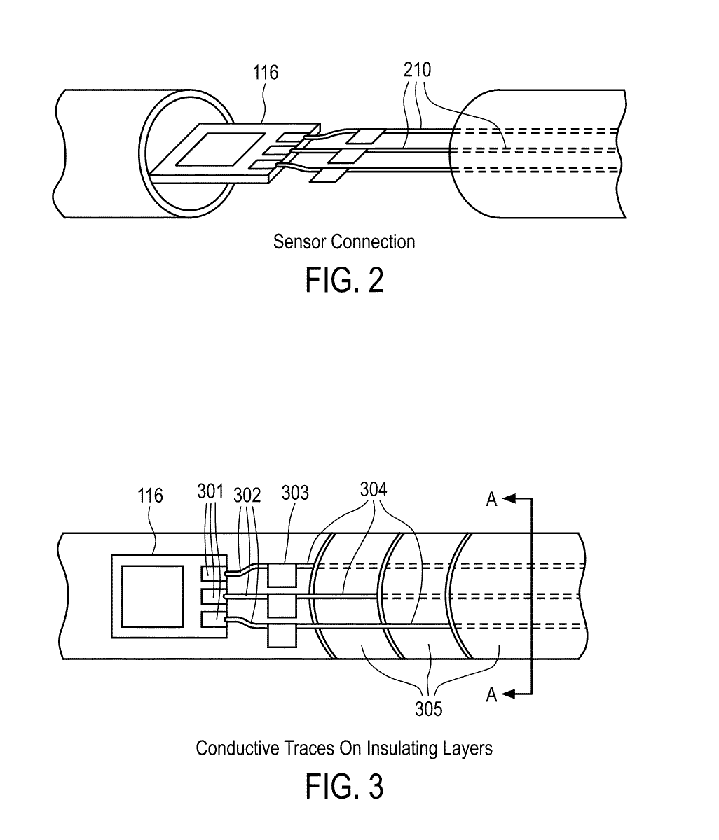

FIG. 2 shows a close up view of the sensor 116 with the electrical transmission conductors 210. These conductors terminate at the electrical contacts 112 at the proximal end of the pressure wire 110. With this construction, the mating connector, whether in the form of a connector 140, or in the form of a wireless transceiver 145 is located at the proximal end of the pressure wire 110 where the electrical contacts 112 are located on the pressure wire 110.

This arrangement for the wireless transceiver 145 can be an impediment to the work flow as transceiver should be smaller and light weight and ideally should perform like a torque device. A torque device, not shown, also needs to be able to be positioned anywhere proximal to where the pressure wire exits the human body and not be constrained to the proximal end or a particular fixed location.

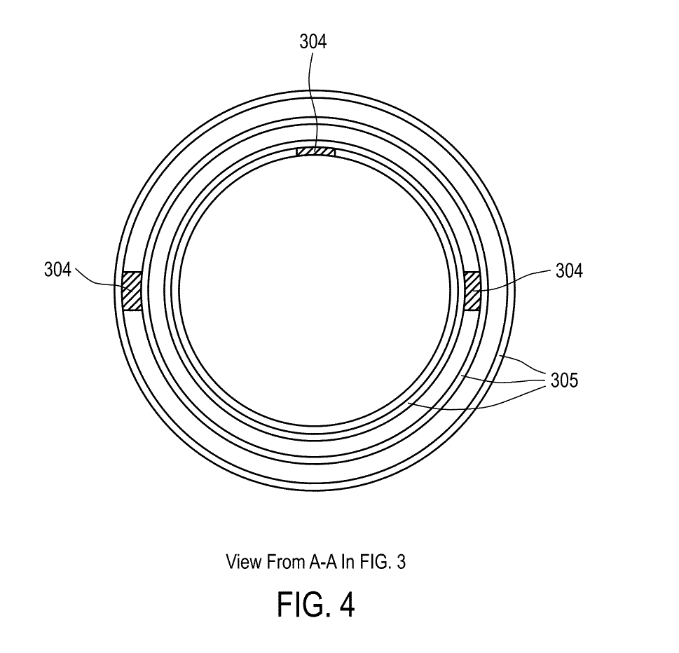

Referring to FIG. 3, the conductors that electrically connect the sensor to the equipment for acquisition, processing and display have been replaced with electrically conductive traces, 304, embedded in insulating layers 305. Three such insulating layers are illustrated in FIG. 3.

In some embodiments, the traces are terminated in pads 303, which are connected to pads 301, on the sensor chip via wire bonding with gold wires 302. Other connection schemes known to persons skilled in the art are also possible.

The traces 304 are distinguished from one another by the number of insulating layers 305 as well as the circumferential locations as indicated in the cross-sectional representation in FIG. 4.

Shielding layers, not shown, can also be implemented to improve the electrical performance of these conductive traces if needed.

These traces 304 can be metallization via various depositing process or conductive polymer and the insulating layers 305 can be various insulating polymers, like polyimide film.

It is also possible to print conductive polymer onto an insulating substrate and achieve similar results. Beside these additive processes, it is also possible to start with a conductive layer on top of an insulating layer, subtractive processes can then be used where the conductive material is removed to result in conductive traces remaining on the insulating layer to serve as conductors.

It is possible to have variations along this theme. For example, multiple conductive traces can reside in the same layer underneath one insulating layer if they can be separated adequately apart. This may be an advantage in the case of multiple sensor chips. One sensor chip can have its conductive traces residing in one layer, while the other can have its conductive traces in another layer.

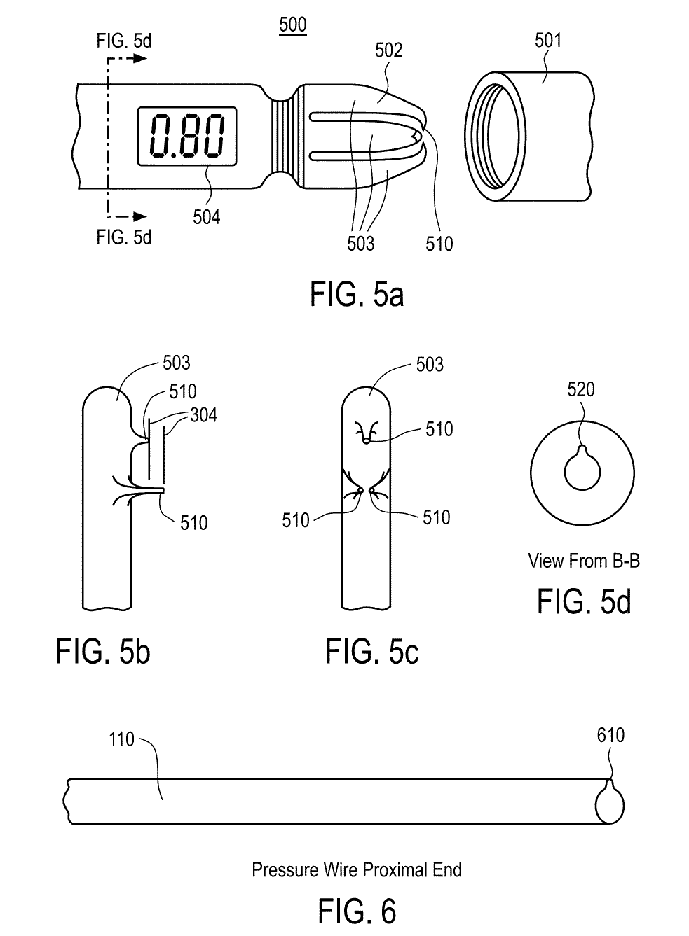

In FIG. 5a, an exemplary torque device 500, is shown with a cap 501 and collet 502, an arrangement where as the cap is advanced, the fingers 503 of the collet 502 will close on and grip on the pressure wire 110. Pressure wire 110 is not shown.

Different ways to implement a torque device are possible.

In FIG. 5a, some of the fingers have a tapered tip 510, capable of penetrating the insulation layers 305, and making contact with the appropriate traces 304, thereby forming electrical connection(s). Different shape and arrangement for the finger 503 to make electrical contacts with the conductive traces 304 are also possible.

Different fingers 503 can have different length tapered tip 510 capable of penetrating to the correct depth to make contact with the conductive trace 304 through the various insulating layers 305.

FIGS. 5b and 5c show two close up views of one embodiment of a finger with a tapered tip configuration designed to simultaneously penetrate two insulating layers 305 to make contact with conductive traces 304 lying at two different depths.

The configuration is such that while making contact with the deeper layer, it avoids shorting with the shallower layer.

This implementation of the tapered tips is useful where multiple sensor chips 116 are present at the distal end of the pressure wires and the conductive traces are embedded in separate layers at different depths. Different length tapered tip 510 can engage different sensor chip signals at different depth levels with no ambiguity. Even if the number of conductive traces is small enough to fit with in the circumference of a single layer of insulating layer, it may still be advantageous to keep the number of fingers 503 small but utilize multiple tapered tips 510 to engage the conductive traces at different depths. Such flexibility is provided for in these embodiments.

Other configurations and methods for the tapered tips to engage the conductive traces are also possible.

In FIG. 5d, a view from B-B of FIG. 5a, the body of the collet 502 has a guiding track 520 to guide the insertion of the torque device such that the orientation of the fingers 503 remain aligned with the conductive traces 305 correctly. In FIG. 6, the portion of the pressure wire 110 that accepts the torque device has a corresponding guiding ridge 610 that allows the torque device to slide along it once the guiding track 520 is aligned with the guiding ridge 610.

This is one example of a mechanical means to ensure a proper orientation of the torque device. Using a visible strip marking on the guide wire for aligning with a counterpart marking on the torque device is an example of a visual means for achieving correct alignment.

Other ways to provide orientation guidance are known for those skilled in the art.

In FIG. 5a, a display 504 is also shown, where result derived from the sensor can be made available to the user of the torque device.

This torque device being able to make electrical connection with the sensor 116 can now provide the needed signal acquisition, processing and wireless transmission to a receiver outside the sterile area of the catheterization laboratory.

In this embodiment, it is important to make the transceiver unit small and light weight as well as being able to position freely along a much larger range in the proximal portion of the pressure wire and behave like a torque device.

To achieve this behavior, some parts of the acquisition and processing are partitioned off the transceiver 145 and locate on the pressure wire body proper. The constraint is to maintain the profile such that the diameter of the entire pressure wire can still accept delivery of other device designed to be delivered over a guide wire, e.g. balloon and stent, usually 0.014 inch in diameter.

In one embodiment, a piece of signal processing component can be interposed and embedded in the envelope of the proximal portion of the pressure wire such that a partially processed signal emerges on the continuation of a conductive trace.

In another embodiment, multiple such interposed segments can be implemented in the proximal portion of the pressure wire in order to reduce the size and weight of the transceiver 145 to better perform like a torque device.

In another embodiment, transceiver 145 only sends out the processed results for display without the pressure signals derived from the sensor chip 116.

The proximal portion of the pressure wire 110 is more tolerant of having any stiff sections that are required to implement signal conditioning and processing components. These components are being off-loaded from the torque device to enable a smaller form factor for the torque device that also doubles as a transceiver.

Note that this proximal portion of the pressure wire does not enter the human body.

In a modern catheterization laboratory, many pieces of equipment vie for the limited space available around the sterile patient table. Able to provide a minimally invasive pressure measurement device that conforms as much as possible to other interventional device like a balloon improves the work flow immensely.

As all the communication between the sensor chip and the torque device takes place in between the insulating layers and the conductive traces, the pressure sensing can also be implemented in the form of a stand-alone sleeve that is delivered over the preferred guide wire that the user has chosen.

This approach of performing the pressure measurement differs from the approach of implementing a pressure wire. The advantage of this approach is that the operator can use his preferred guide wire without any possible compromise on the wire performance but with the possible disadvantage that an additional catheter, however small, needs to be delivered over the guide wire and subsequently removed to allow for other device to be delivered over the same guide wire again for the next steps in the procedure.

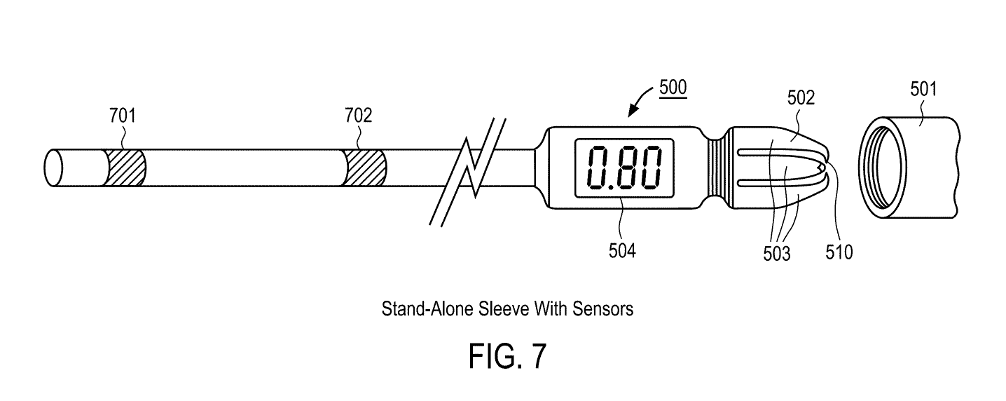

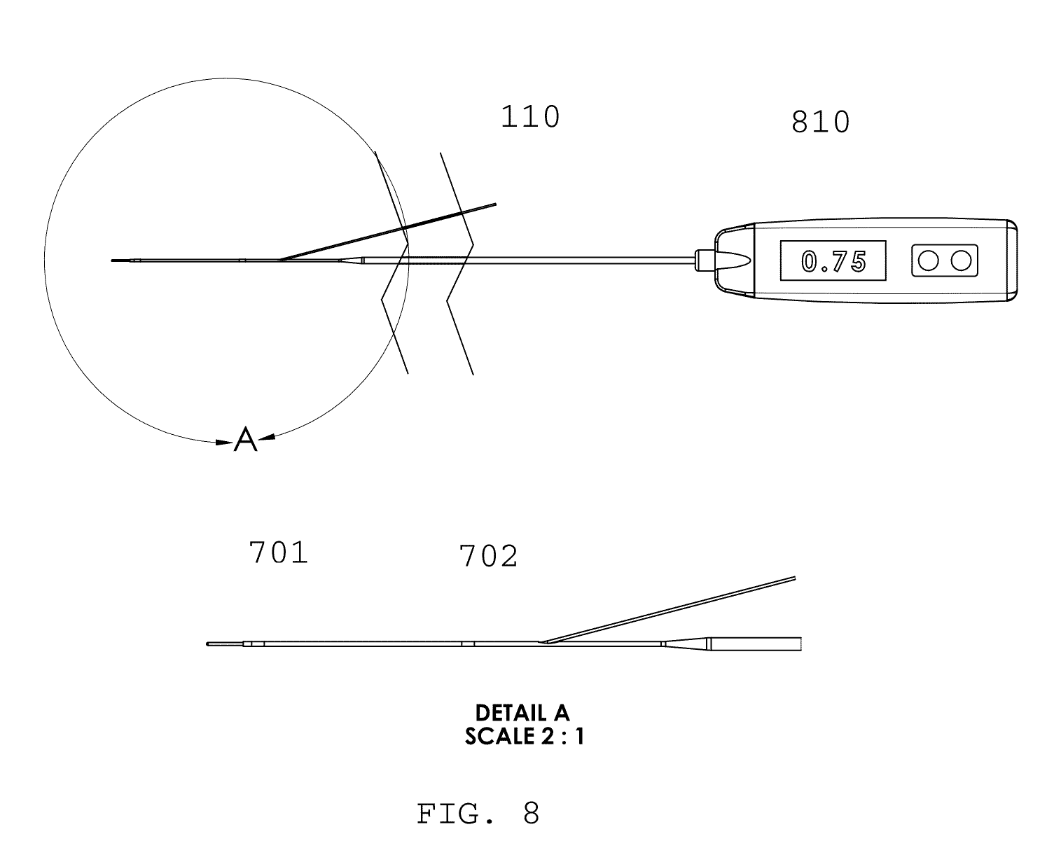

FIG. 7 illustrates the concept of this embodiment where sensor 701 and sensor 702 are located on a sleeve and are in communication, wireless or wired, with torque device 500. A display 504 is also shown on the torque device 500. This torque device 500 can also optionally communicate, via a wireless receive 146, with equipment 150 with its display 154 and input device 156 or a stand alone remote display.

In one embodiment, sensors 701 and 702 are wireless. Sensor 701 is distal to a stenosis in a coronary artery, sensor 702 is in the aorta. Together, they provide two independent pressure measurements that are transmitted to the torque device 500. The display 504, on the torque device can then, as an example, display the measured Fractional Flow Reserve value which is a ratio of the mean of the distal pressure over the mean of the proximal pressure.

In one embodiment, the torque device 500 itself can activate the two sensors, 701 and 702, as indicated in FIG. 7. Sensor 701 is deployed distal to a stenosis in the coronary artery while sensor 702 remains in the aorta such that upon activation by the torque device via an electromagnetic wave, they send out their respective pressure measurement signals wirelessly. These signals are received by the torque device and any computation result based on these two measurement signals is then shown on the display 504. No other capital equipment in required and both pressure signals needed to generate the ratio for Fractional Flow Reserve (FFR) is obtained simultaneously without the need for a pullback.

It is also possible to implement sensor using MicroElectroMechanical Systems (MEMS) technology and they can be piezo-resistive or capacitive in their principle of operation. It is also possible to implement the sensor using piezo-electric polymer or ceramic.

The use of piezo-electric polymer is of particular value since it does not require the use of rigid sensor chip and can be conformable to the shape of a guide wire geometry.

The choice of the specific sensor technology for 701 and 702 depending on process complexity and cost of manufacturing with corresponding pro's and con's.

It should be appreciated that it is possible to have a hybrid system where the sensors 701 and 702 can have wired connections between them and then wirelessly communicate with torque device via wireless means. This has a certain advantage when the pressure sensing is implemented as a stand alone device to be delivered over an existing guide wire. Sensor 702 which resides in the aorta as opposed to the coronary artery would have more room to accommodate a wireless transceiver to transmit both pressure measurements. This will then not impact the need to have a small form factor in the distal sensor 701 to have accurate pressure measurement.

In one embodiment, the sensor 701 is implemented with a piezo-electric polymer that generates a voltage when experience a change in pressure. The capacitance of sensor 701 can also be a function of pressure as it changes dimension. This voltage or capacitance change is measured via conductive traces or other wired transmission means to a proximal sensor 702 which resides in the aorta. Sensor 702 itself senses pressure at the aorta as well as handling any needed conditioning and processing of pressure signal from sensor 701 and together wirelessly provides the result or partial result to the torque device 500 on its display 504.

It is contemplated that this invention is applicable to physiological parameters other than pressure. One characteristics of this invention is the use of a low cost, disposable transceiver. It can be made small if the data rate and power consumption are low—which dictates the kind of information and type of signal acquisition and processing that can be accomplished.

Physiologic parameters like pressure, temperature, pH value, etc., are slow varying parameters that can be acquired with low sampling frequency, simple processing, if any, and low data transmission rate. The power consumption is also correspondingly low.

The improvement described here affords a better torque transmission as it removes the need to have a lumen to accommodate the electrical or optical transmission lines. In particular, the electrical connection scheme also improves the electrical performance as the parasitic capacitance is reduced by increasing the separation of the transmission lines. The improved construction also allows for better integration of multiple sensors.

The improvement with a wireless transfer of the physiologic signal allows for a more compatible operation with how a guide wire is used in the PCI procedure. A wireless embodiment also improves the work flow and avoids the need to have a large instrument near the patient's bed during the procedure. Wireless communication between the sensor and the torque device also makes for a compact system when a simple display on the torque device is adequate for the procedure.

Multiple sensors eliminate the need to perform a pullback procedure to obtain pressure information from multiple locations.

A stand alone embodiment allows pressure measurement with an existing primary guide wire and eliminates the need for a wire exchange procedure.

Several variations of the stand alone sleeve with multiple sensors as illustrated by FIG. 7 are possible. For example, the distance between the two sensors, 701 and 702, can be made variable to accommodate different lesion locations in the coronary arteries while keeping the proximal sensor in the aorta.

The sleeve can also be constructed such that a guide wire exit port allows for a rapid exchange catheter configuration as described in U.S. Pat. No. 5,451,233 “Angioplasty Apparatus Facilitating Rapid Exchanges” by Paul Yock, which is incorporated by reference for all purposes.

The sleeve in the above configuration can now have a catheter handle, as opposed to a torque device, where a larger display can be accommodated. This larger display can display both waveforms and numerical results from processing of the waveforms.

In this configuration, as shown in FIG. 8, the connection between the sensors (701, 702) and the electronics in the handle, 810, will not require embedding the conductors in insulating layers and are self contained within the stand-alone sleeve catheter.

Having the sensors implemented on the sleeve itself allows for integration with other interventional devices that could benefit from a pressure measurement to monitor the progress of the interventional procedure. For example, if this pressure measuring sleeve is integrated with a Chronic Total Occlusion (CTO) device, the pressure monitoring can indicate when the CTO device has succeeded in entering the distal true lumen as opposed to entering a false lumen in the intima of the vessel wall. This can reduce the use contrast medium and radiation from the angiogram.

Other applications can include integration with percutaneous valve implantation where the reduction of the pressure gradient across the valve is an important parameter. Having a sleeve approach for pressure measurement allows for relatively easy integration with such percutaneous valve devices.

An exemplary embodiment of the vascular pressure measurement device having a small outer diameter is described below.

It is beneficial to ascertain first the need for the small outer diameter for the vascular pressure measurement device.

In many cases performing Percutaneous Coronary Intervention (PCI) procedures requires delivering the procedural device via a guide wire that has been deployed across a lesion only after a significant effort due to tortuosity and bending of the blood vessels and obstruction of the blood vessel lumen due to one or more lesions. In such cases, it is essential that the sleeve used in this procedure would maintain a smaller outer diameter in order to easily cross those obstructing lesions.

In some intervention procedures, the sleeve is implemented in a rapid exchange format where only a short segment of the sleeve accommodates the guide wire. By loading the sleeve onto the guide wire from the proximal end of the guide wire, a single operator can deliver the catheter. The rapid exchange implementation is sometimes referred to as a “monorail” implementation and the lumen that accommodates the guide wire is then referred to as the guide wire engagement rail.

Furthermore, it is sometimes desired to integrate the sleeve with electronic components, such as sensors, to allow for making physiological measurements, as is the case of pressure measurements. Additionally, the sleeves may carry electrical transmission lines. This will further increase the crossing profile of the sleeves if a sensor is implemented on the sleeve and the sleeve also serves as the guide wire engagement rail as is sometimes desirable to do.

Thus, it is critical that the sleeve that contains sensor 701, the distal sensor, and 702, the aortic sensor, used in a blood pressure measurement, has a small outer diameter.

In some intravascular blood pressure measurements, the measurement is done with a pressure wire of the same geometrical form factor as a standard guide wire, for example, 0.014″ diameter. It is then desirable that a sleeve over a standard guide wire does not increase the overall diameter significantly, for example, an overall diameter of the sleeve of approximately 0.018″ would be suitable. This will help to ensure that measurements taken with a sleeve over a standard guide wire are comparable to those taken with a pressure wire of the size of the standard guide wire.

As an example, if a pressure wire is of a diameter of about 0.014″, and a sleeve with sensors are to be implemented such that the overall profile were not to exceed approximately 0.018″, the wall thickness for the sleeve would typically need to be less than 0.001″ taken into account that the inner diameter of the sleeve needs to be about 0.016″ or 0.017″ to accommodate the about 0.014″ standard guide wire.

Since the sleeve has to accommodate standard guide wires, with the desire to have as small as practical outer diameter of the sleeve, there is a need for designing a sensor and its required electrical transmission lines into a sleeve with as thin a wall thickness as possible.

Sleeves are often fabricated from extruded tubing. Sometimes, extrusion places a minimum limit on the wall thickness of the extruded tubing. Many polymers cannot be extruded to too thin a wall thickness especially with inner diameters that are often encountered in intravascular blood pressure measurements.

Another challenge with a tubular structure is the difficulty in creating a conductive surface or an insulating surface on the inner wall of the tubular structure. As a rule of thumb, some deposition techniques can penetrate up to 40 times the opening diameter. Using the about 0.017″ inner diameter as an example, this will allow a length of less than about 0.68″ (1.7 cm) along the inner wall of the tubular structure which may be inadequate in some applications.

In one embodiment of the invention, a planar approach to build up tubular structures that can achieve small wall thickness is illustrated. Furthermore, these tubular structures can also include various insulating and conductive elements for fabricating sensors and other electronic components. Thus, as an example, it will be possible to build sensors 701 and 702 on a sleeve that will serve as guide wire engagement rail while measuring blood pressure as described above.

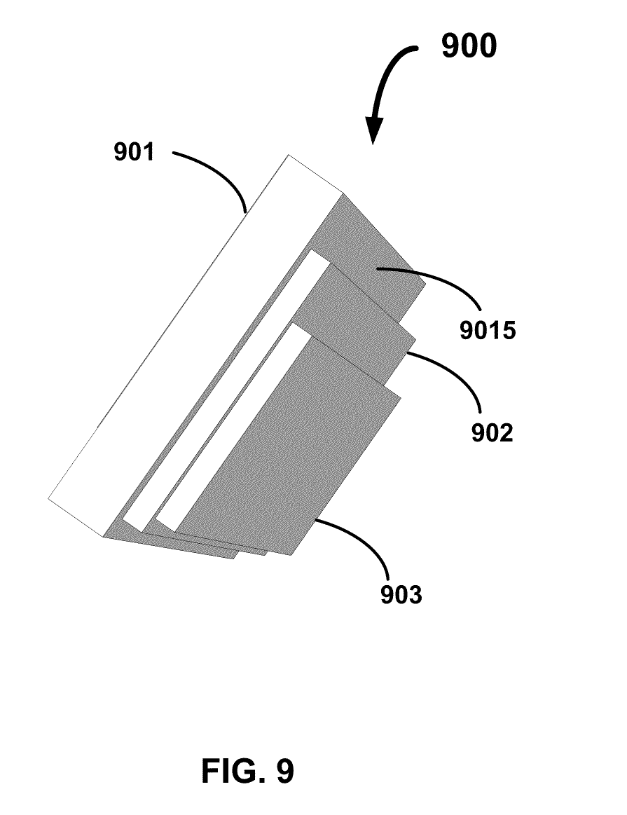

FIG. 9 illustrates a planar structure 900 wherein a dielectric layer 901 can serve as a substrate for layers to be added to it. A conductive layer 902 is coated on a surface 9015 of the dielectric layer 901. An insulating layer 903 is coated on top of the conductive layer 902. The conductive layer 902 can serve as an electrode for sensors 701 (not shown) or 702 (not shown). The insulating layer 903 can serve as an insulating layer between the electrode and a fluid flowing in the sleeve (not shown). The fluid may be blood if the sleeve is used in intravascular blood pressure measurement.

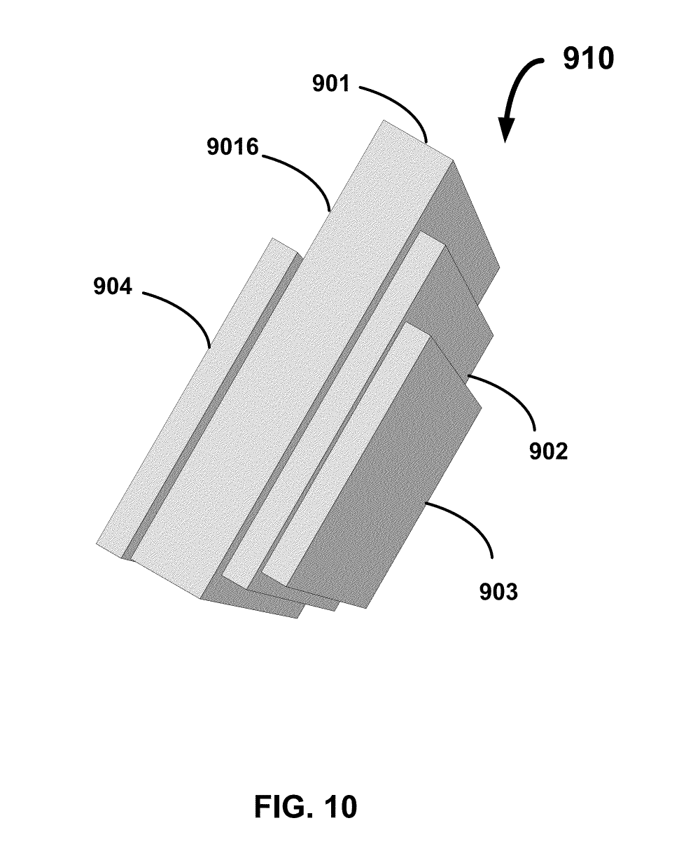

FIG. 10 illustrates a planar layered structure 910 wherein a second conductive layer 904 can be added on the other side of the dielectric layer 901, on surface 9016. The length of the second conductive layer 904 can determine the length of an electrode of the pressure sensor 701, the distal sensor, in the embodiment described earlier. Note that the sensor 702 has a more forgiving thickness constraint as it serves to measure the aortic pressure and can remain in the guiding catheter or outside the coronary artery and would not need to cross the lesion being interrogated.

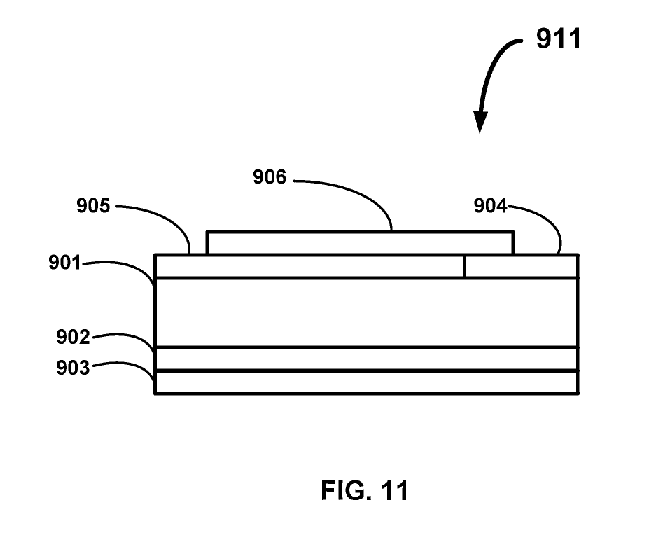

FIG. 11 illustrates a layered construct 911 wherein a second insulating layer 905 is added to the dielectric layer 901 on the surface 9016. The second insulating layer 905 is added on the surface 9016 of the dielectric layer 901 but without covering the conductive layer 904. The second conductive layer 904 can be utilized as an electrode. Thus, adding a third conductive layer 906 on top of the second conductive layer 904 and the second insulating layer 905 can serve to connect to the second conductive layer 904 to the proximal end of the sleeve where, as described above, the thickness constraint is more forgiving. This facilitates accessing both sensors as this is also where the proximal sensor 702 is located.

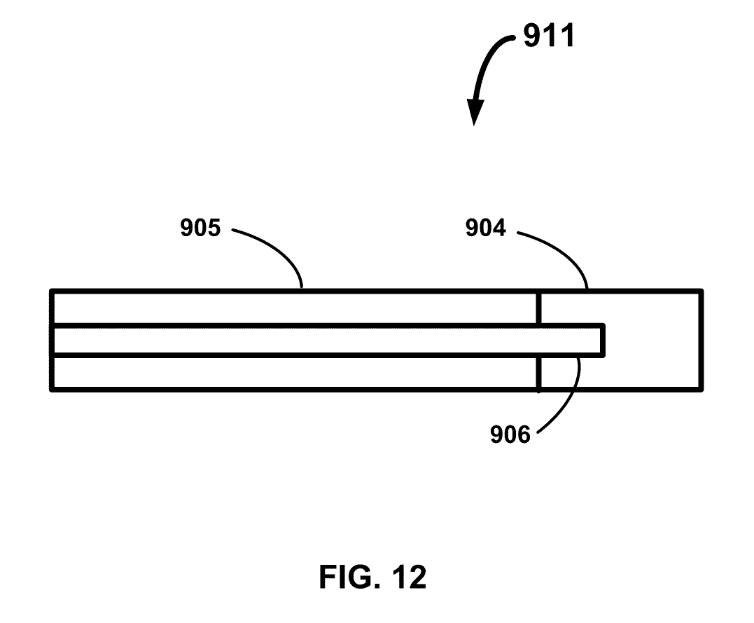

FIG. 12 provides a top view for the layered construct 911 showing how the third conductive layer 906 can be configured to provide the electrical connection to the distal sensor 701.

There are numerous ways in which the layered construct 911 can be used to build up complex scheme of conductive transmission lines with insulating layers and shielding layers. With the right masking, different width or shapes of the various layers or traces can be obtained.

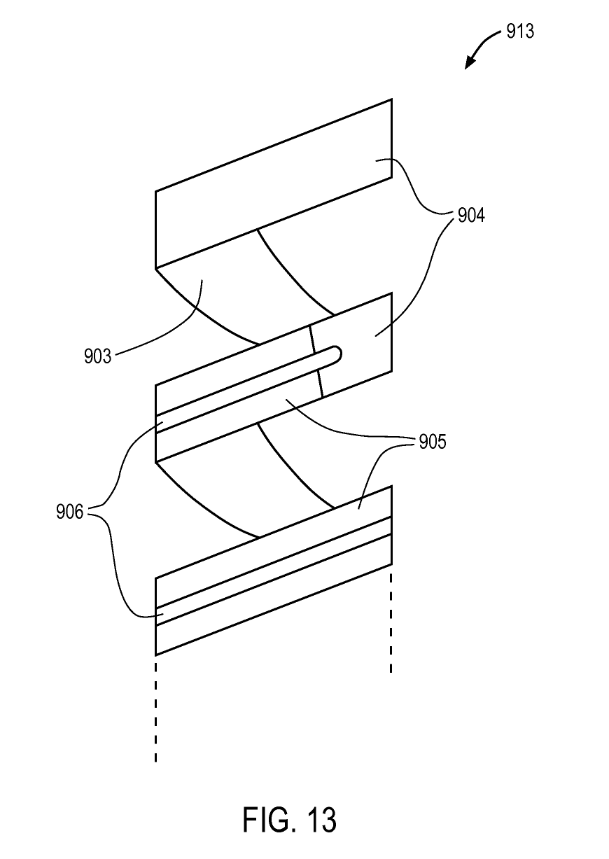

The fabricated layered construct 911 can be made in strip form and wound in a helical fashion to achieve a helical construct 913 shown in FIG. 13. The helical construct 913 can serve as a sleeve with the needed conductive, insulating and shielding surfaces built up layer by layer. This helical construct 913 can be configured as the sleeve carrying the sensors 701 and sensor 702 (not shown). The gap shown between the helical turns is for illustrating the under layers.

In the helical construct 913, the conductive layer 902, not shown, is underneath the insulating layer 903.

In the helical construct 913, the conductive layer 902, not shown, can serve as a source electrode to the sensor 701. The third conductive layer 906 can serve as a return transmission line to the sensor 701, sensor 701 is defined by conductive layer 904.

The helical structure 913 has the advantage that it can be implemented in making the source electrode shared between the sensor 701 and the sensor 702 which otherwise would be quite difficult to create a common conductive surface spanning the distance between the two sensors if the dielectric is in the form of a small tube.

Sharing a common source electrode between the two sensors 701 and 702 is significant as that would reduce the number of conductive connections needed to access the distal sensor 701. Thus both sensors 701 and 702 can be accessed at the proximal end where space constraint is relatively reduced.

Different variations of the helical structure 913 are possible. For example, the source electrode can be placed on the outside of the helical surface while the return electrode can be located inside the helical surface.

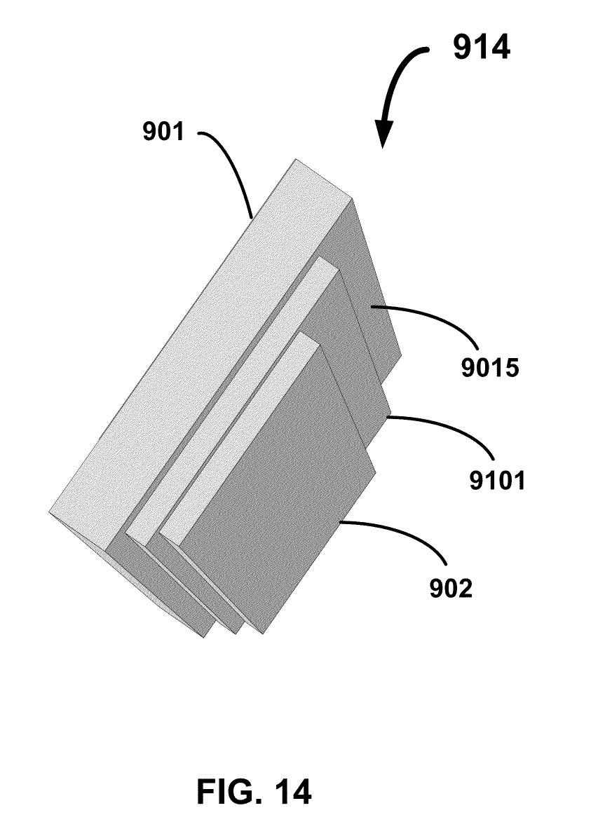

In another embodiment, illustrated in FIG. 14, a layered structure 914 is shown whereby an inserted insulating layer 9101 is deposited on the dielectric layer 901 before depositing the conductive layer 902. This may be necessary since often times when a very thin dielectric is used, there may be pin holes defects that can create a short circuit between the common source electrode and return electrode. The inserted insulating layer 9101 serves to address this situation.

Other advantages for adding the inserted layer 9101 can be the flexibility in the choice of the properties of this inserted layer such as the enhancement of the adhesion of the deposited conductive layer 902 to the dielectric layer 901 and improve the matching of properties between the dielectric layer 901 and the conductive layer 902 such as matching the thermal expansion coefficients for both layers 901 and 902. This last advantage can be particularly useful in certain procedures done at different temperatures. For example, crossing a Chronic Total Occlusion (CTO) may involve ablation energy such that ambient temperature surrounding the sensor may change while it is desirable to monitor the pressure change as an indication of achieving patency of the lumen.

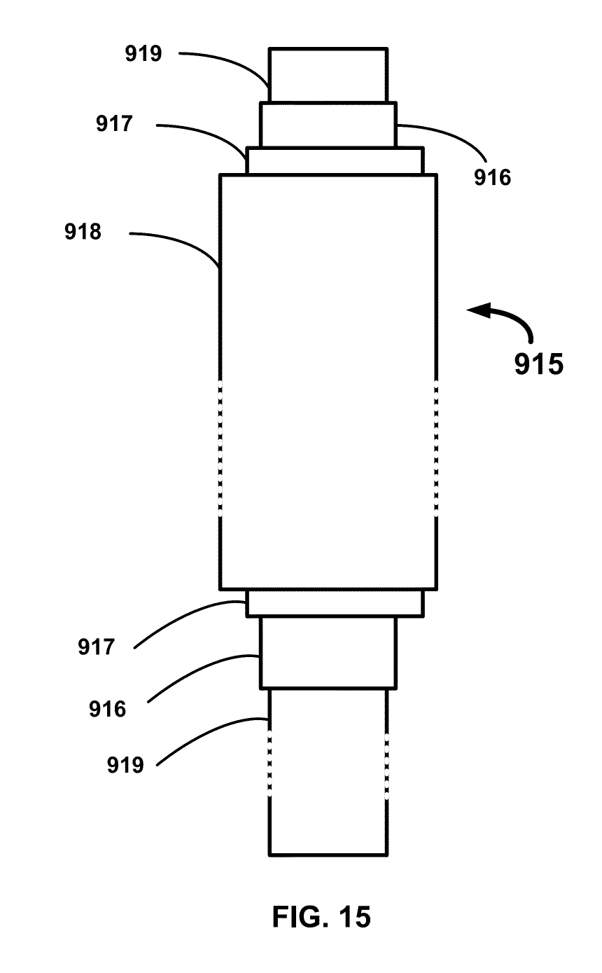

In yet another embodiment, the sensors 701 and 702 can be constructed in a tubular form 915 which can be built, as illustrated in FIG. 15, by alternately applying an insulating layer 916, a conductive layer 917 and a dielectric layer 918 in a sequence to affect the building of the tubular form 915 which can then be further built up into a pressure sensor. Structure 915 may be achieved by applying the insulating layer 916, the conductive layer 917 and the dielectric layer 918 in a sequence onto a mandrel 919 such as a spindle or an axle used to support materials being deposited. Then removing the mandrel 919 after all layers are deposited, leaving a remaining to have the tubular form 915 with minimal wall thickness configured to have a desired diameter. Structure 915 can also be built up by the “missing wax” or “lost wax” technique(s) whereby the supporting tubular material, 919, has low melting point, as is with certain alloy(s), or dissolved with solvents, such that the tubular form can be obtained.

While this invention has been described in terms of several embodiments, there are alterations, modifications, permutations, and substitute equivalents, which fall within the scope of this invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A measuring system for measuring at least one physiological parameter of a human, the measurement system comprising:
an elongated sleeve having a compact diameter configured to be delivered over a standard guide wire and further configured to be flexibly threaded into a tortuous or a diseased vascular pathway of a human; and
at least one sensor operatively coupled to a distal end of the sleeve, and wherein the at least one sensor is configured to measure at least one physiological parameter at at least one location inside the human.

2. The measuring system of claim 1 wherein the sleeve has an outer diameter that is approximately 20 mils or less and capable of accommodating the standard guide wire.

3. The sleeve of claim 2 wherein the standard guide wire has a diameter of approximately 14 mils.

4. The measuring system of claim 1 wherein the sleeve has a wall thickness of approximately 2.5 mils or less.

5. The measuring system of claim 1 wherein the sleeve is formed by winding a planar layered structure into a helical construct.

6. The measuring system of claim 1 wherein the sleeve is formed by depositing a sequence of layers on a mandrel.

7. The measuring system of claim 6 wherein the sequence of layers includes at least one conductive layer and at least one insulating layer.

8. The measuring system of claim 7 wherein the at least one conductive layer and the at least one insulating layer alternate.

9. The measuring system of claim 1 wherein the sleeve is formed by depositing a sequence of layers deposited using a lost wax technique.

10. The measuring system of claim 9 wherein the sequence of layers includes at least one conductive layer and at least one insulating layer.

11. The measuring system of claim 10 wherein the at least one conductive layer and the at least one insulating layer alternate.
